(19) United States
(12) Plant Patent Application Publication
(10) Pub. No.: US 2009/0089907 P1
(43) Pub. Date: Apr. 2, 2009

(54) Doritaenopsis plant named "Sunrise Beautifil Girl"

(76) Inventor: Hou-Chih Lin, Taipei City (TW)

Correspondence Address: BIRCH STEWART KOLASCH & BIRCH, PO BOX 747, FALLS CHURCH, VA, 22040-0747, US

(21) Appl. No.: 11/905,455

(22) Filed: Oct. 1, 2007

(51) Int. Cl. A01H 5/00

(52) U.S. Cl. PLT/311

(57) ABSTRACT

A new and distinct Doritaenopsis orchid plant named ‘Sunrise Beautiful Girl’ having attractive and unique purple-colored flowers with a very clear white edge and economical propagation via tissue culture. ‘Surnise Beautiful Girl’ has upright, sturdy flowering stems and excellent postproduction longevity.

### BOTANICAL CLASSIFICATION

Doritaenopsis sp.

### VARIETY DENOMINATION

‘Sunrise Beautiful Girl’.

The present invention relates to botanical classification/cultivar designation: Doritaenopsis Orchid cultivar Sunrise Beautiful Girl.

### BACKGROUND OF THE INVENTION

The present invention comprises a new and distinct cultivar of Doritaenopsis Orchid, hereinafter referred to by the cultivar name, Sunrise Beautiful Girl.

The new cultivar is a product of a planned breeding program conducted by the inventor in Taiwan. The objective of the breeding program is to create new uniform pot-type Doritaenopsis Orchid cultivars having attractive flower coloration.

The new cultivar breeder was Hou-Chih Lin. The new cultivar was discovered by the inventor from within the progeny of a cross-pollination of two identified proprietary selections of Doritaenopsis Orchid, not patented, on Mar. 3, 1994, in a controlled environment in Taiwan. Later, it was verified and registered in Royal Horticulture Society (R.H.S.) and had its variety name “Dtps. Sunrise Beautiful Girl” on January, 2003.

Asexual propagation by tissue culture in a laboratory in Taiwan has been used to increase the number of plants for evaluation and has demonstrated that the unique combination of characteristics as herein disclosed for the new Doritaenopsis Orchid are firmly fixed and are retained through successive generations of asexual reproduction.

### SUMMARY OF THE INVENTION

The following traits have been repeatedly observed and are determined to be basic characteristics of new cultivar which, in combination, distinguish this Doritaenopsis Orchid as a new and distinct cultivar:

1. Petal main color: purple; Petal color pattern: white edge.

2. Purple-colored flowers with a very clear white edge and greyed purple-colored labellum.

3. Freely flowering habit.

4. Upright, sturdy flowering stems.

5. Excellent postproduction longevity.

Plants of the new cultivar differ primarily from plants of the parent cultivars in flower color.

Plants of the new Doritaenopsis Orchid can be compared to plants of the cultivar Dtps. Ruey Lih Beauty, differing from plants of the cultivar Dtps. Ruey Lih Beauty the following characteristics:

1. Plants of the new Doritaenopsis Orchid are larger than plants of the cultivar Dtps. Ruey Lih Beauty.

2. Plants of the new Doritaenopsis Orchid are more freely flowering than plants of the cultivar Dtps. Ruey Lih Beauty.

3. Plants of the new Doritaenopsis Orchid have more flowers per inflorescence than plants of the cultivar Dtps. Ruey Lih Beauty.

4. Plants of the new Doritaenopsis Orchid have larger flowers than plants of the cultivar Dtps. Ruey Lih Beauty.

5. Plants of the new Doritaenopsis Orchid and the cultivar Dtps. Ruey Lih Beauty differ in flower color. The color of petal and sepal are purple and bright with a very clear white edge.

### BRIEF DESCRIPTION OF THE DRAWINGS

Colors in the photographs may appear different from the color values that appear in the detailed botanical description which more accurately describe the new cultivar.

FIG. 1 is a side view of a plant of ‘Sunrise Beautiful Girl’ flowering in a 12 cm pot.

FIG. 2 is a close-up view showing the characteristics of the flower.

FIG. 3 is a close-up view showing the characteristics of the leaf.

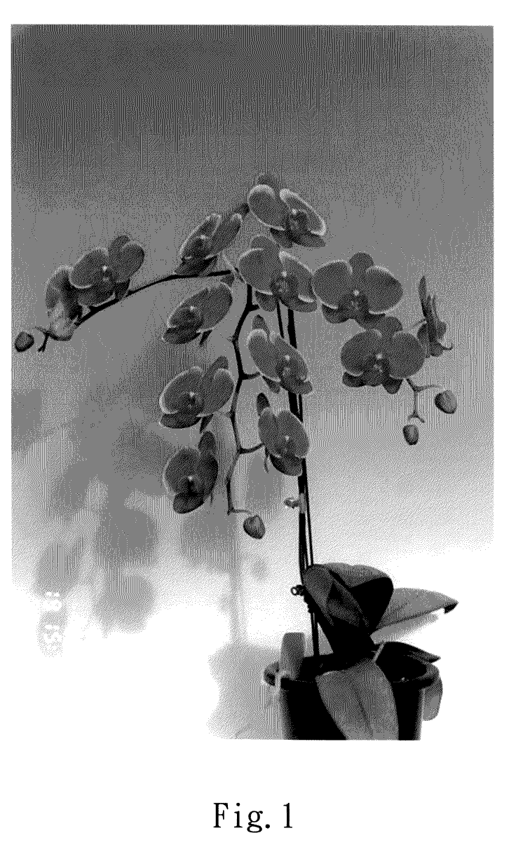

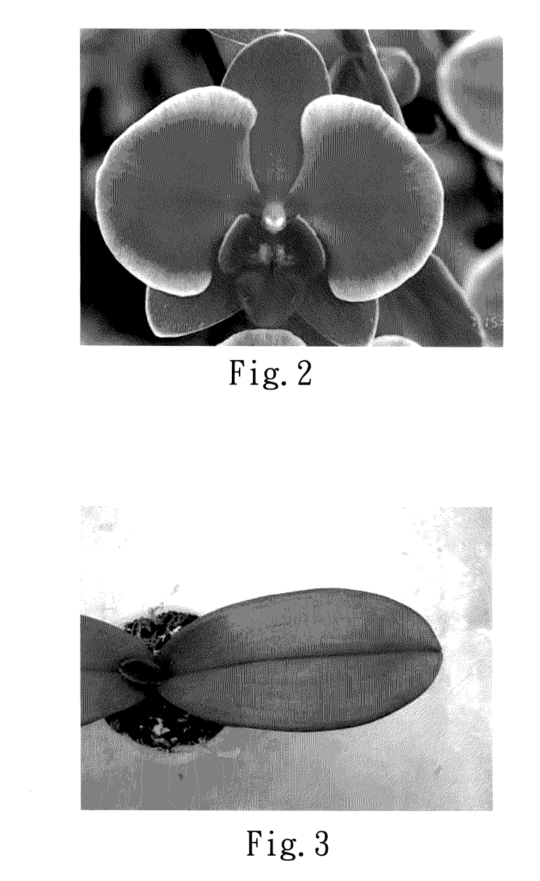

### DETAILED DESCRIPTION OF THE PREFERRED EMBODIMENTS

Plants of the new cultivar have not been observed under all possible environmental conditions. The phenotype may vary significantly with variations in environment such as temperature and light intensity, without however, any change in genotype. In the following description, color references are made to The Royal Horticultural Society Colour Chart, 1995 Edition, except where general terms of ordinary dictionary significance are used.

Plants used for the aforementioned photographs and following detailed botanical description were 14 months old and grown in 12 cm containers in Taiwan, in a greenhouse with day temperatures about 25 to 28° C., night temperatures about 18 to 20° C., and light levels about 15,000 to 25,000 lux. The photographs and the detailed botanical description were taken during the winter.
Parentage:
   Seed.—Dtps. Formosa Rose, not patented.
   Pollen.—P. Hsinton Rose, not patented.
   Propagation.—Asexual propagation by tissue culture.
   Root description.—Very thick, fleshy, and greenish white in color.
   Plant shape.—Two-ranked leaves affixed to a short central stem (monopodial growth). Single flowers arranged on upright and sturdy flowering compound racemes.
   Plant height, soil level to top of foliar plane.—About 14 to 17 cm.
   Plant height, soil level to top of inflorescences.—About 60 to 70 cm.
   Plant diameter.—About 35 to 45 cm.
   Flowers per stem.—Approximately 10 to 15.
Foliage description:
   Leaves.—Simple, opposite, sessile.
   Quantity per plant.—About 5 to 6.
   Length.—About 20 to 25 cm.
   Width.—About 8 to 10 cm.
   Shape.—Oblong.
   Apex.—Obtuse.
   Base.—Cuneate.
   Margin.—Entire.
   Aspect.—Mostly flat and folded upward from the midrib.
   Texture, upper and lower surfaces.—Leathery, thick, glabrous.
   Venation.—Parallel.
   Color (upper surface).—RHS 146A.
   Color (lower surface).—RHS 146A.
Flower description:
   Flower type.—Single zygomorphic flowers, orbicula in shape.
   Flower arrangement.—Compound racemes.
   Flowering stems.—Upright, freely branching and sturdy.
   Flowering habit.—Plants freely flowering; plants typically produce one to two branched flowering stems with at least 10 to 15 flowers each.
   Fragrance.—Flowers not fragrant.
   Self cleaning or persistent.—Flowers persistent.
   Flower aspect.—Flowers facing mostly outward.
   Natural flowering season.—From January to April in Taiwan. Plants begin flowering about 14^{th} months after planting.
   Post-production longevity.—Plants of the new Doritaenopsis Orchid maintain good leaf and flower substance for about three to six months on the plant under interior environmental conditions. Cut flowers of the new Doritaenopsis Orchid maintain good substance for about three to four weeks.
   Inflorescence length.—About 50 to 60 cm.
   Inflorescence diameter.—About 25 to 30 cm.
   Flower bud (just before anthesis).—
   Flower bud shape.—bullet-like.
   Flower bud length.—About 2.8 cm.
   Flower bud diameter.—About 2.5 cm.
   Flower bud color.—About RHS N77B.
   Flower diameter.—About 8 to 10 cm.
   Flower depth.—About 1.6 to 1.9 cm.
   Number of petals.—Two per flower.
   Petal size.—Lateral petals similar in size and shape; the lowermost petal, the labellum, is deeply three-lobed.
   Length.—About 4 cm.
   Diameter.—About 4 cm.
   Shape.—Semicircular.
   Apex.—Rounded.
   Base.—Attenuate; fused with the column.
   Margin.—Entire.
   Texture.—upper and lower surfaces: Velvety, silky; smooth.
   Color.—When blossoming, the main color of the adaxial surface of petal is purple (RHS 78A). The main color of the abaxial surface of petal is purple (RHS N74B).
Labellum:
   Length, not flattened.—About 3.4 cm.
   Diameter, not flattened.—About 1.8 cm.
   Shape.—Deeply three-lobed with two prominent callosities on the upper surface at the central junction of the lateral lobes and base of the midlobe. The lateral lobes fold upward about the column, the extends extend forward and is are terminated by two twisted filiform appendages (about 14 mm in length) at the apex.
   Base color of the abaxial surface of the apical lobe.—Purple (RHS N79C).
   Tip color of the adaxial surface of the apical lobe.—Purple (RHS N79C).
Sepals:
   Quantity.—Three per flower.
   Dorsal sepal length.—About 4.9 cm.
   Lateral sepal length.—About 3.6 cm.
   Dorsal sepal diameter.—About 3.6 cm.
   Lateral sepal diameter.—About 3.1 cm.
   Shape.—Elliptical.
   Apex.—Rounded acute to retuse.
   Base.—Attenuate; fused with the petals and column.
   Margin.—Entire.
   Texture, upper and lower surfaces.—Velvety; smooth.
   Dorsal sepal main color.—RHS78A.
   Dorsal sepal pattern color.—RHS 78A.
   Lateral sepal main color.—RHS78A to the base.
   Lateral sepal pattern color.—spotted RHS 78A and shaded RHS 155C.
Peduncles:
   Length.—About 45 to 60 cm.
   Diameter.—About 7 mm.
   Aspect.—Upright.
   Strength.—Strong, sturdy.
   Texture.—Smooth, glabrous.
   Color.—RHS 147A.
Pedicels:
   Length.—About 20 to 25 cm.
   Diameter.—About 4 to 5 mm.
   Aspect.—About 45° from vertical.
   Strength.—Strong.
   Texture.—Smooth, glabrous.
   Color.—RHS 147A.
   Color towards the base.—RHS 147A.
Reproductive organs: The stamens, style and stigmas are fused into a single, short structure called the column, possessing one terminal anther with pollen grains united into a pollinia, which are covered by an anther cap. The stigma is located under the column behind the pollinia. The ovary is inferior with three carpels presented. The plant has not produced seed.
Column:
   Length.—About 1 cm.
   Diameter.—About 6 mm.
   Color.—RHS 78A.
   Color towards to apex.—RHS 78A.
Pollinia:
   Quantity of pollen masses.—Two pollen masses.
   Diameter.—About 1 mm.
   Color.—RHS 23A.
Ovary:
   Length.—About 4.9 to 5.3 mm.
   Diameter.—About 3 to 6 mm.
   Color.—RHS 15C.
Root: In tissue culture plantlets, the first root emerged 30 days after being deflasked.
   Diameter.—About 5 mm.
   Color.—RHS 144B.
Disease/pest resistance: Resistance to known pathogens and pests common to Doritaenopsis Orchids has not been observed on plants of the new cultivar grown under commercial greenhouse conditions.
Temperature tolerance: Plants of the new Doritaenopsis Orchid have been observed to be tolerant to temperatures from about 14 to 32° C.

What is claimed is:

1. A new and distinct cultivar of Doritaenopsis plant named ‘Sunrise Beautiful Girl’, as illustrated and described.
